(19) United States
(12) Patent Application Publication
(10) Pub. No.: US 2018/0000657 A1
(43) Pub. Date: Jan. 4, 2018

(54) APPLICATOR FOR FEMININE CARE PRODUCT

(71) Applicant: The Procter & Gamble Company, Cincinnati, OH (US)

(72) Inventors: Gustavo Jose Camargo-Parodi, Mason, OH (US); John Ferrer, Mason, OH (US); Benjamin Philip Goodman, Cincinnati, OH (US)

(21) Appl. No.: 15/443,382

(22) Filed: Feb. 27, 2017

Related U.S. Application Data
(60) Provisional application No. 62/301,241, filed on Feb. 29, 2016.

(51) Int. Cl. A61F 13/20; A61F 13/26

(52) U.S. Cl. CPC A61F 13/2097; A61F 13/26

(57) ABSTRACT

A method of designing a feminine hygiene applicator is disclosed. The method includes obtaining a first applicator; obtaining onset crystallization temperature for the first applicator; obtaining a second applicator; obtaining L*, a*, b* values for the second applicator; configuring a third applicator comprising an onset crystallization temperature which is within 3 percent of the onset crystallization temperature of the first applicator; configuring the third applicator to have an L* value within 20 percent of the L* value for the second applicator; configuring the third applicator to have an a* value within 20 percent of the a* value for the second applicator; and configuring the third applicator to have a b* value within 20 percent of the b* value for the second applicator.

### FIELD OF THE INVENTION

The present invention relates generally to an array of applicators having preferred onset crystallization temperatures and opacity values, more specifically to applicators for feminine hygiene products.

### BACKGROUND OF THE INVENTION

Feminine care products, such as tampons and pessaries, are generally used by women within the vagina, such as, e.g., to absorb menstrual or other body exudates, for pelvic support, and/or for other feminine needs. Such feminine products can be inserted into the vagina digitally, such as, e.g., by using a finger, or can be inserted into the vagina by using an applicator.

Applicators typically comprise an insertion member and a plunger. The material to be expelled from the applicator, such as an absorbent tampon or pessary, can be positioned within the insertion member. The insertion member can have a first end for insertion of the material and a second end for receipt of the plunger. To use the applicator, the consumer will grasp the insertion member, position the first end appropriately, such as, e.g., into the body, and move the plunger in the insertion member towards the first end to insert the material.

Based on the foregoing, fit between the plunger and the insertion member are very important. If the fit between the plunger and the insertion member is too tight, the force needed to expel the material from the insertion member can dramatically increase. In contrast, if the fit between the insertion member and the plunger is too loose, the plunger could disengage from the insertion member. This could potentially lead to an unsanitary condition, e.g. if the plunger falls on the floor.

Applicators can be made up of different materials. For example, currently available in market there are applicators which comprise cardboard and applicators which comprise plastic. Applicators are provided in a myriad of colors. However, the move from one color of applicator to a different colored applicator is far from trivial. Particularly for plastic applicators, the introduction of a new/different pigment to produce the different color can modify the shrinkage rate of the plastic in the applicator. Unfortunately, this modification can negatively impact the fit between the insertion member and the plunger.

Based on the foregoing, it would be desirable to provide an array of applicators comprising differing colors which have similar dimension and fit. It would also be desirable to provide a method of producing applicators of different colors and/or a method of changing applicator colors from a first color without negatively impacting the dimensional fit between parts. Also, it would be beneficial to provide an applicator having a color with acceptable dimensions.

### SUMMARY OF THE INVENTION

A method of designing a feminine hygiene applicator is disclosed. The method includes obtaining a first applicator; obtaining onset crystallization temperature for the first applicator; obtaining a second applicator; obtaining L*, a*, b* values for the second applicator; configuring a third applicator comprising an onset crystallization temperature which is within 3 percent of the onset crystallization temperature of the first applicator; configuring the third applicator to have an L* value within 20 percent of the L* value for the second applicator; configuring the third applicator to have an a* value within 20 percent of the a* value for the second applicator; and configuring the third applicator to have a b* value within 20 percent of the b* value for the second applicator.

A method of designing a feminine hygiene applicator is additionally disclosed. The method includes obtaining a first applicator; obtaining onset crystallization temperature for the first applicator; obtaining a second applicator; obtaining L*, a*, b* values and an opacity value for the second applicator; configuring a third applicator comprising an onset crystallization temperature which is within 3 percent of the onset crystallization temperature of the first applicator; configuring the third applicator to have an L* value within 20 percent of the L* value for the second applicator or an a* value within 20 percent of the a* value for the second applicator and/or a b* value within 20 percent of the b* value for the second applicator; and configuring the third applicator to have an opacity value which is less than that of the opacity value for the second applicator.

### BRIEF DESCRIPTION OF THE DRAWINGS

FIG. 1 is a plan view of an applicator comprising a barrel and a plunger.

FIG. 2 is a DSC curve with Calculated Onset Temperature of Crystallization (OTX).

### DETAILED DESCRIPTION OF THE INVENTION

As used herein, “array” means a display of packages comprising disposable articles of different sizes having like article constructions (e.g., same elastomeric materials [compositionally and/or structurally] in the flaps, graphic elements) said packages having the same brand and/or sub-brand, and said packages oriented in proximity to each other in a given area of a retail store. An array is marketed as a line-up of products normally having like packaging elements (e.g., packaging material type, film, paper, dominant color, design theme, etc.) that convey to consumers that the different individual packages are part of a larger line-up. Arrays often have the same brand, for example, “Always,” and same sub-brand, for example, “Radiant.” A different array may have the brand “Always” and the sub-brand “Pearl.” Furthermore, the packaging may be distinctly different. Arrays also often have the same trademarks, including trademarks of the brand, sub-brand, and/or features and/or benefits across the line-up. “On-line Array” means an “Array” distributed by a common on-line source.

As used herein, the term “feminine care product” includes absorbent articles useful for feminine needs, such as articles that typically can be intended for feminine use internally, such as, e.g., within a user's vagina. Internal feminine care products can include, for example, tampons and pessaries.

As used herein, the term “pessary” refers to any type of substantially non-absorbent structure for the purpose of reducing urine leakage and/or supporting a prolapsed uterus and/or bladder. Such pessaries can have any variety of shapes and sizes including cylinder, ovate, spherical, tubular, annual rings, “U” shaped, cup shaped, rings, cubes or donut shaped, and can function in any suitable manner, such as, e.g., by direct application of support, lever force, expansion of the device by selection of material, and/or by inflation of the device.

As used herein, the term “vaginal canal” refers to the internal genitalia of the human female in the pudendal region of the body. The terms “vaginal canal” or “within the vagina” as used herein are intended to refer to the space located between the introitus of the vagina (sometimes referred to as the sphincter of the vagina) and the cervix.

As used herein “applicator” refers to a device or implement that facilitates the insertion of a tampon, medicament, pessary, treatment device, visualization aid, or other into an external orifice of a mammal, such as the vagina, rectum, ear canal, nasal canal, or throat. Non-limiting specific examples of such include any known hygienically designed applicator that is capable of receiving a tampon may be used for insertion of a tampon, including the so-called telescoping, tube and plunger, and the compact applicators, an applicator for providing medicament to an area for prophylaxis or treatment of disease, a spectroscope containing a microcamera in the tip connected via fiber optics, a speculum of any design, a tongue depressor, a tube for examining the ear canal, a narrow hollow pipe for guiding surgical instruments, and the like.

As used herein, “compression” refers to the process of pressing, squeezing, compacting or otherwise manipulating the size, shape, and/or volume of a material to obtain a tampon having a vaginally insertable shape. The term “compressed” refers to the state of a material or materials subsequent to compression. Conversely, the term “uncompressed” refers to the state of a material or materials prior to compression. The term “compressible” is the ability of a material to undergo compression.

The term “cross-section” as used herein, is any 5 mm thick section of the tampon orthogonal to the longitudinal axis.

As used herein, “fluid wicking” refers to the ability of a material to carry fluid or moisture by capillary action. The fluid wicking capacity of a medium may be measured by grams of fluid drawn per gram of tampon weight over a fixed period of time.

The term “folded” as used herein, is the configuration of the tampon pledget that may be incidental to lateral compaction of the absorbent material or may purposely occur prior to a compression step. Such a configuration is readily recognizable, for example, when the absorbent material abruptly changes direction such that one part of the absorbent material bends and lies over another part of the absorbent material.

As used herein, “generally cylindrical” refers to the usual shape of tampons as is well known in the art, but which also includes oblate or partially flattened cylinders, curved cylinders, and shapes which have varying cross-sectional areas (such as a Coke™ bottle shape). The longitudinal axis refers to the longest linear dimension of the tampon. The cross-section refers to a slice taken at right angles to the longitudinal axis.

As used herein, the term “insertion end” refers to the portion of the tampon or applicator including the end that is intended to enter the vaginal canal first when inserting the tampon or applicator into the vaginal canal.

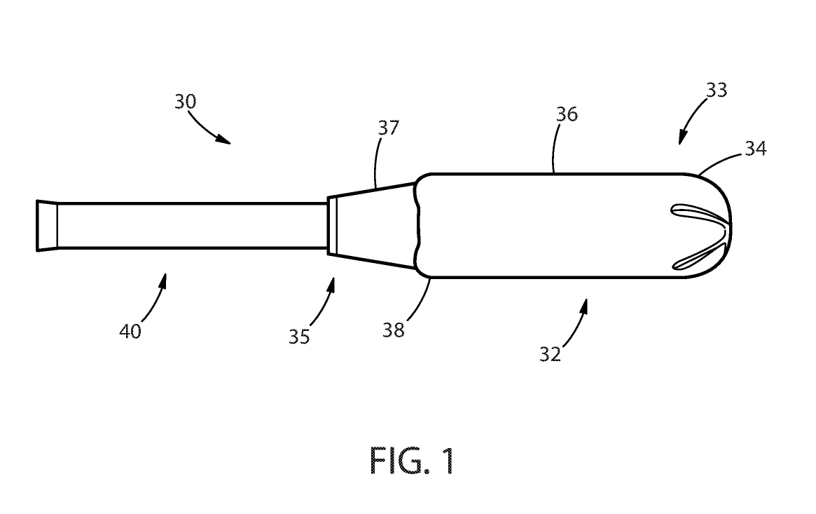

As used herein, the term “longitudinal axis” of a tampon refers to the axis that runs through the center of the tampon as shown in FIG. 1. A portion of the tampon may be asymmetric about the longitudinal axis, such as when the withdrawal end region is flared and distorted from the original shape of the rest of the tampon (such as a “fin shape”). Further, the longitudinal axis may be linear or non-linear.

As used herein, “overwrap” refers to the liquid pervious material covering the exterior surface of the absorbent member. The overwrap may permeate the inner region of a compressed absorbent member. The overwrap may extend below the withdrawal end to form a skirt portion. The overwrap may be fluid wicking. The overwrap, as defined herein, may possess a horizontal wicking capacity of at least about 2, alternatively from about 3 to about 6 grams of fluid per gram of tampon at a 500 second interval. Suitable overwraps are disclosed in greater detail in U.S. Pat. No. 6,840,927 and U.S. Pat. No. 7,112,192, filed Nov. 18, 2002, entitled “Tampon With an Overwrap or Overwraps Having Both Masking and Wicking Properties,” issued to Hasse, et al.

As used herein, the term “tampon,” refers to any type of absorbent structure that is inserted into the vaginal canal or other body cavities for the absorption of fluid therefrom, to aid in wound healing, or for the delivery of active materials, such as medicaments, or moisture.

The “outer surface” of a tampon refers to the visible surface of the (compressed and/or shaped) tampon prior to use and/or expansion. At least part of the outer surface may be smooth or alternatively may have topographic features, such as ribs, spiraling ribs, a mesh pattern, or other topographical features. Typically, tampons are constructed from an absorbent material, which has been compressed and/or shaped in any or all of the width direction, the radial direction, and the axial direction, in order to provide a tampon which is of a size and stability to allow insertion within the vagina or other body cavity.

As used herein, the term “radial axis” of a tampon refers to the axis that runs at right angles to the longitudinal axis of the tampon as shown in FIG. 1.

The term “rolled,” as used herein, is the configuration of the tampon pledget after winding the absorbent material upon itself.

The term “vaginally insertable shape” as used herein refers to the geometrical form of the absorbent tampon after compression. The tampon may be compressed into a generally cylindrical configuration in the radial direction along the longitudinal and/or lateral axes, axially, or in both the radial and axial directions. An example of a typical compressed tampon may be one which may be about 10-16 mm wide and about 40-50 mm long depending on the level of absorbency. While the tampon may be compressed into a substantially cylindrical configuration, other shapes are possible. These may include shapes having a cross section that may be described as rectangular, trapezoidal, seni-circular, hourglass, or other suitable shapes.

As used herein, the term “withdrawal end” refers to the portion of the applicator opposite the insertion end.

As used herein, “cm” is centimeter, “mm” is millimeter, “g” is gram, “gsm” is grams per meter squared, “dpf” is denier per fiber, “g/g” is gram of fluid per gram of sample, “wt” is weight, “psi” is pound per square inch.

While particular embodiments have been illustrated and described, it would be obvious to those skilled in the art that various other changes and modifications can be made without departing from the spirit and scope of the invention.

The array of applicators described herein use two or more polymer resins having an onset crystallization temperature that is within 0.4 degrees Celsius of the other polymer resins. Each of the applicators has an opacity of greater than 50%. The applicators may be a typical “tube and plunger” type arrangement and may be plastic or other suitable material. Additionally, a “compact” type applicator is also suitable. Where the tampon is shaped and provides aesthetic appeal to consumers, it is may be desirable to combine the shaped tampon with an applicator type which enables the user to observe at least a portion or the whole shape of the shaped tampon. Two techniques which allow the user to better notice the shape of the tampon are to either make visual observation possible through the use of a translucent or even transparent applicator materials, or to provide a tampon applicator insertion end that better follows and hence better displays the profiled shape of the enclosed shaped tampon than the typical commercial tampon applicators comprising straight-walled cylindrical inserter tubes often made from molded plastic or laminated cardboard tubes. The applicator may be flushable as described in U.S. Pat. No. 6,730,057, filed Mar. 16, 2001, entitled “Flushable Tampon Applicators,” issued to Zhao, et al. The applicator may be corrugated as described in U.S. Pat. No. 7,066,870, filed Jun 25, 2002, entitled “Method of Producing a Corrugated Tampon Applicator,” issued to Fedyk, et al.

The applicator may have a grip region as described in U.S. Pat. Nos. 8,303,558; 7,081,110; 8,449, 491; or 8,075,512. The applicator may have an absorbency indicator as described in U.S. Pat. No. 7,166,101, filed Dec. 9, 2005, entitled “Tampon Outer Surface Having Increasing Number of Written Identifiers to Indicate Absorbency,” issued to Denti, et al. The grip region may have visual indicia. Any visual indicia suitable from distinguishing the grip portion from the barrel portion and/or the plunger can be used, such as, e.g., color, such as, e.g., a contrasting color and/or a coordinating color, sheen, such as, e.g., a glossy or matte finish, shimmer, any type of mark, figure, picture, identification code, symbol, icon, pattern, text, such as, e.g., a word, number, nomenclature, sentence, or instruction, line, line segment, curved line, band, arrow, area of coloration, or any other printed indicia having a purpose of providing a signal or guide to the user.

The tube or barrel portion can be constructed from any suitable material. Suitable materials include, for example, any combinations thereof, polyethylene, polypropylene, polybutylene, polystyrene, polyvinylchloride, polyacrylate, polymethacrylate, polyacrylonitrile, polyacrylamide, polyamide, nylon, polyimide, polyester, polycarbonate, polylactic acid, polyhydroxyalkanoate, ethylene vinyl acetate, polyurethane, silicone, thermoplastic starch, trans-poly isoprene, derivatives thereof, copolymers thereof, mixtures thereof, or any suitable smooth plastic material. Examples of suitable materials are disclosed in, e.g., U.S. Pat. Nos. 5,346,468 and 5,558,631. In certain embodiments, additives can be included in the material to alter or enhance certain material properties. Suitable additives include, for example, mold release agents, slip agents, surface energy modifiers, inorganic fillers and/or any other suitable additives. In certain embodiments, the barrel portion can be coated with a substance to give it a high slip characteristic, such as, e.g., with wax, polyethylene, a combination of wax and polyethylene, cellophane, clay, and other lubricants that can facilitate comfortable insertion.

The barrel portion can be sized and configured to house a feminine hygiene product, such as, e.g., an absorbent tampon and/or pessary. In certain embodiments, the size of the barrel portion can be determined primarily by the dimensions of the feminine hygiene product. For example, the barrel portion can have inner diameters of about 5.0 millimeters to about 22.0 millimeters and a wall thickness of about 0.2 millimeter to about 2.0 millimeters. The inner diameter of the barrel portion can be greater than the diameter of the feminine hygiene product to prevent the barrel portion from interfering with the expulsion of the feminine hygiene product from the barrel portion. In certain embodiments, the inner diameter of the barrel portion can have varying diameters and shapes to conform to the profiled shape of the enclosed feminine hygiene product, such as, e.g., a tampon. The barrel portion can have a length sufficient to house the feminine hygiene product prior to the expulsion of the feminine hygiene product from the applicator into the vagina.

The barrel portion can be of any suitable cross-sectional shape. In certain embodiments, the barrel portion can include a generally non-circular cross-sectional shape, such as, e.g., oval, rectangular, elliptical, oblate, or other suitable shapes. The barrel portion can have a cross-sectional shape that has a greater thickness than width or vice versa. In certain embodiments, the barrel portion can have a substantially uniform cross-section, such as, e.g., having the same cross-section along the length. In other embodiments, the barrel portion can have varying cross-sectional shapes and/or cross-sectional sizes, such as, e.g., a barrel portion having a smaller cross-sectional area near the insertion end of the barrel and a larger cross-sectional area near the opposite end.

The insertion end of the barrel portion can be open-end or closed-ended. In certain embodiments, the insertion end of the barrel portion can include petals, corrugations, pleats, a film cap, or other means for covering the barrel portion prior to expulsion of the tampon. In certain embodiments, the material, such as, e.g., a feminine care product can be loaded into the barrel portion prior to covering the insertion end of the barrel portion. Alternatively, the insertion end of the barrel portion can be covered prior to loading the feminine hygiene product into the barrel portion.

The plunger can be constructed from any suitable material. The barrel portion can be constructed from any suitable material. Suitable materials include, for example, paper, paperboard, cardboard, cellulose, such as, e.g., molded cellulose, or any combinations thereof, polyethylene, polypropylene, polybutylene, polystyrene, polyvinylchloride, polyacrylate, polymethacrylate, polyacrylonitrile, polyacrylamide, polyamide, nylon, polyimide, polyester, polycarbonate, polylactic acid, polyhydroxyalkanoate, ethylene vinyl acetate, polyurethane, silicone, thermoplastic starch, trans-poly isoprene, derivatives thereof, copolymers thereof, mixtures thereof, or any suitable smooth plastic material. Suitable plungers are disclosed in, e.g., U.S. Pat. Nos. 5,346,468 and U.S. Pat. No. 5,558,631. In certain embodiments, additives can be included in the material to alter or enhance certain material properties. Suitable additives include, for example, mold release agents, slip agents, surface energy modifiers, pearlescent agents, inorganic fillers, and/or any other suitable additives.

The plunger can be hollow or solid. In certain embodiments, the plunger can have a hollow interior, a first end, and a second end opposed to the first end. The first end is the portion of the plunger that pushes against the tampon during the expulsion of the tampon from the barrel portion. The plunger may be unitary. The second end is the portion of the plunger in which the axial force is applied to expel the tampon from the barrel portion. In certain embodiments, the plunger can have a locking mechanism, such as, e.g., a locking mechanism that retains the plunger within the barrel portion and/or grip portion of the applicator prior to depression of the plunger and expulsion of the tampon. Examples of such locking mechanisms are described in, for example, U.S. Pat. Nos. 6,019,744 and 6,450,986. The plunger may comprise an outer sleeve and an inner sleeve, the inner sleeve capable of slidingly engaging with the outer sleeve.

In certain embodiments, at least a portion of the applicator can contact and/or conform to at least a portion of the surface of the tampon. Rigid insertion end structures can be shaped in a suitable manner, such as, e.g., by injection molding, or by reshaping in a secondary process to provide at least a degree of profiled shape observation. Alternatively, insertion ends of applicators made from flexible or pliable materials, such as films, paper and flexible wovens or non-wovens, can also be used. Such flexible or pliable insertion ends include those which partially or fully enclose the tampon comprising a “sleeve” or a “tube,” such as, e.g., in U.S. Pat. Nos. 2,922,422 and 2,922,423; a “sheath,” such as, e.g., in U.S. Pat. Nos. 2,092,427 and 3,749,093; a “barrel,” such as, e.g., in U.S. Pat. No. 5,135,475; a “bag,” such as, e.g., in U.S. Pat. No. 3,358,686; or a “film enclosure,” such as, e.g., in U.S. Pat. No. 4,610,659.

It has been found that applicator fit between the tube or barrel and plunger may be impacted by the color chosen and the opacity of the applicator. Color is created by adding colorants to the base resin. Plastic colorants are compounds of typically 3-5 pigments plus additives in a resin or liquid carrier. The biggest hurdle of any colorant development is to develop a colorant that processes well and that does not impact the technical specifications of the molded applicator. These colorant may act as contaminants to the base resin and therefore impact various properties of the resin that may impact the applicator tolerances.

Changing the color of an injected-molded part in a semi-crystalline polymer changes the dimensions of the parts produced. Pigments, among other additives, may act as nuclei for crystal growth. However, different colorants may have different efficiency. The different colorants may and create different crystal structures that manifest themselves as dimensional differences between colors. These dimensional differences can result in unacceptable performance of the part or an assembly of parts.

One of the possible manifestations is shrinkage by the polymer. Shrinkage is inherent to polyolefin processing and results from crystallization/re-arranging of polymer chains on cooling. More shrink occurs in the flow direction which can lead to considerable internal stresses within the molded applicator. The type of pigment (size, shape and coating) may impact the amount of shrinkage. White (primarily TiO2 pigment) is close to the un-pigmented natural base resin and creates little shrinkage. However, the greens and blues are often the worst colors exhibiting superior heat resistance and also very high shrinkage due their pigment size and shape.

One possible measure that relates to part dimensions is the onset crystallization temperature of the applicator resin. The onset crystallization temperature is a direct measure of the nucleating efficiency because nucleating agents change the activation energy required before crystallization can begin, and the onset crystallization temperature is the energy level at which the activation energy has been satisfied. The onset crystallization temperature can impact the shrinkage rate of the resin as it cools. Further, modifying the onset crystallization temperature can impact the shrinkage rate. Once an applicator mold is created for a first resin, that mold may or may not create a second applicator with the desirable dimensional tolerance if a different resin is used even though the same applicator mold was used to create both applicators. However, it has been found that by controlling the onset crystallization temperature, one may use different resins in a mold and achieve a final applicator with similar dimensions and fit.

Further, by matching the onset crystallization temperature of the different resins, the opacity of a given applicator may be increased while still using the same mold to create the first applicator using a first resin and the second applicator using a second resin.

Further, by matching the onset crystallization temperature of the different resins, one may have an array of feminine care products having a first resin first color and a second resin second color while still using the same mold and creating applicators with essentially the same dimensions. For example, by matching the onset crystallization temperatures so that the delta is less than 0.4 C., one can use a resin having a first color that has an a* color value between −128 and 128 and a b* color value between −128 and 128 to create a first applicator and a second resin having a second color that has an a* color value between −128 and 128 and a b* color value between −128 and 128 to create a second applicator. The first color a* color value may be between 0 and 128. The first color and the second color may have a delta a* color value of less than 20.

TABLE 1

| Sam- | | | | | | |
|---|---|---|---|---|---|---|
| ple | Sample | OTX | | Min. OTX | Opacity | |
| ID | Description | Avg | Std Dev | Delta | Avg | Std Dev |
| R | Tampax Pocket | 110.71 | 0.056 | n/a | 93.1 | 3.03 |
| | Pearl Blue | | | | | |
| | Regular | | | | | |
| A | Tampax Pearl | 108.57 | 0.485 | 0.07 | 75.3 | 2.92 |
| | Light Blue Super | | | | | |
| B | Invention Appli- | 108.64 | 0.056 | | 50.2 | 3.11 |
| | cator, Blue Super | | | | | |
| C | U by K Sleek | 108.68 | 0.225 | 0.42 | 85.3 | 2.03 |
| | Blue Regular | | | | | |
| D | U by K Sleek | 109.59 | 0.174 | | 93.0 | 1.13 |
| | Green Regular | | | | | |
| E | U by K Sleek | 109.17 | 0.298 | | | |
| | Purple Regular | | | | | |
| F | U by K Click | 109.21 | 0.848 | 0.97 | | |
| | Fuchsia Regular | | | | | |
| G | U by K Click | 108.24 | 0.097 | | | |
| | Green Regular | | | | | |
| H | Equate Light | 106.64 | 0.288 | 0.18 | 52.5 | 2.3 |
| | Green Regular | | | | | |
| I | Equate Light | 106.82 | 0.091 | | 40.7 | 1.23 |
| | Blue Regular | | | | | |
| J | DG Health Light | 107.02 | 0.042 | n/a | 87.4 | 1.27 |
| | Blue Regular | | | | | |

As shown in Table 1, Sample B and Sample A have an onset crystallization temperature delta of less than 0.4 for an applicator that is the same size. It has been found that by having a onset crystallization temperature delta of a first resin to a second resin of less than 0.4 degrees Celsius, such as, for example, between 0.01 and 0.4, between 0.02 and 0.3, between 0.05 and 0.2 and between 0.07 and 0.4 degrees Celsius, that one may utilize the same mold to create applicators of the same size with either the first or the second resin while maintaining dimensions within the desirable tolerance levels. As shown in Table 1, the onset crystallization temperature may be less than 110 degrees Celsius, such as, for example, below 109 degrees Celsius, below 108 degrees Celsius, or below 107 degrees Celsius. The onset crystallization temperature may be between 107 degrees Celsius and 109 degrees Celsius.

Further one may increase the opacity to greater than 41%, such as, for example, between 41% and 99% opacity, between 41% and 75%, between 41% and 51%, between 60% and 95% opacity, and between 75% and 90% opacity.

An array of feminine care products having a first product having a first applicator and a second product having a second applicator, wherein each of the first applicator and second applicator comprise a barrel portion comprising an insertion end and a grip end disposed opposite the insertion end of the barrel portion; and a plunger slidingly engaged with the barrel portion; wherein the barrel portion and the plunger of each of the first applicator and second applicator comprise a polyolefin resin, wherein the first applicator comprises a first color, wherein the second applicator comprises a second color, wherein each of the first applicator and the second applicator comprise a onset crystallization temperature, wherein the onset crystallization temperature of the first applicator and the onset crystallization temperature of the second applicator differ by less than 0.4 degrees Celcius, and wherein the opacity of each of the first applicator and the second applicator is greater than 41.

The opacity of each of the first applicator and the second applicator may be greater than 41 and less than 75, preferably greater than 41 and less than 51.

The plungers for each of the applicators of the first product and second product may be unitary. The plungers for each of the applicators of the first product and second product may have an outer sleeve and an inner sleeve, wherein the inner sleeve is slidingly engaged with the outer sleeve.

The feminine care product may be selected from the group including tampons and pessaries.

The onset crystallization temperature of each of the first applicator and the second applicator may be less 109 degrees Celsius. The onset crystallization temperature of each of the first applicator and the second applicator may be greater than 107 degrees Celsius.

The first applicator barrel portion may have a first color and the first applicator plunger may have a third color different from the first color. The first color may be an a* color value between −128 and 128 and a b* color value between −128 and 128. The second color may be an a* color value between −128 and 128 and a b* color value between −128 and 128. The first color a* color value and the second color a* color value may differ by less than 20. The first color a* color value may be between 0 and 128.

FIG. 1 is a plan view showing an applicator 30. The applicator 30 comprises an insertion member or barrel 32 and a plunger 40. The applicator barrel 32 has an insertion tip34 proximal a first end 33 and an opposing withdrawal end 35. The applicator barrel region 36 is adapted to contain a feminine care device such as, e.g., a tampon or intravaginal incontinence device (e.g., a pessary). The applicator withdrawal end 35 contains a grip region 37 that may be an indentation region. The grip region 37 may also be demarcated from the barrel region 36, such as, e.g., by one or more shoulder regions 38. Grip regions may comprise three dimensional surface elements that can protrude outward from the grip region.

Typically, tampons are constructed from an absorbent material, which has been compressed in any or all of the width direction, the radial direction, and the axial direction, in order to provide a tampon, which is of a size and stability to allow insertion within the vagina or other body cavity. The tampon is preferably in a so-called ‘self-sustaining’ form, e.g. it will tend to retain its general shape and size, before use. This self-sustaining form need not persist during actual use of the tampon. The tampons herein are typically fluid expanding, e.g. the tampon will expand (or un-compress) upon contact with fluid such as bodily fluids.

The tampon has a top portion, having a topside or top (point) and a bottom side or point, both typically positioned at or forming the ends of the longitudinal axis of the tampon. The top portion of the tampon is typically the portion, which is positioned under the petals, thus typically the part from the top edge of the tube of the applicator to the top of the inserter tip. Because the inserter tip has preferably an opening at the top, part of the op portion of the tampon may be visible through this opening. The tampon has an insertion end and a withdrawal end, whereby the insertion end contains or is typically said top portion, whilst the withdrawal end contains said bottom side.

The tampon may be straight or non linear in shape, such as curved along the longitudinal axis. If the tampon is straight, the length of tampon is the longest distance between the top portion and bottom side and this is generally parallel to or even equal to the longitudinal axis of the tampon. The tampon may be serpentine as described in U.S. Pat. No. 6,824,536, filed May 16, 2002, entitled “Substantially Serpentine Shaped Tampon,” issued to Randall, et al. The tampon may be shaped to have varying perimeters as described in U.S. Pat. No. 6,932,805, filed May 16, 2002, entitled “Shaped Tampon,” issued to Kollwitz, et al. The tampon may be discontinuous as described in U.S. Pat. No. 8,597,267, filed April 18, 2007, entitled “Tampon Having at Least One Physical Discontinuity,” issued to Noel, et al. The tampon may be shaped to have improved aspect ratios when compressed as described in U.S. Pat. No. 8,684,987, filed February 8, 2007, entitled “Self-Orienting Tampon Having Improved Aspect Ratio,” issued to Hasse, et al. The tampon may have an assymetric insertion end as described in U.S. Pat. No. 8,216,202, filed Sep. 22, 2006, entitled “Tampon Having an Asymmetric Insertion End,” issued to Minoguchi, et al.

The tampon may be shaped to have a desired shape after expansion. An example of this is described in U.S. Pat. No. 6,953,456, entitled “Tampon Having An Oval Form After Expansion and Process For Producing the Same,” issued to Fuchs, et al. The tampons may be compressed in a manner that allows for faster expansion and for increased expansion in the width dimension as described in U.S. Pat. No. 6,554,814, filed Oct. 24, 2000, entitled “Protection Tampon and Method of Making,” issued to Agyapong, et al.

The tampon has a width, which may vary in different portions of the tampon. If the tampon is straight, the transverse axis of the tampon is preferably perpendicular to the longitudinal axis and then the tampon width is typically perpendicular to the length. Often, the tampon is typically cylindrical, having preferably an endless sidewall or endless longitudinal side, preferably with a flat bottom side and with a rounded or dome-shaped top portion; then, the width of the tampon corresponds to the largest cylindrical cross-section diameter, and the length corresponds to the longest distance between the bottom side and the top of the rounded portion.

The tampon may have a plurality of recessed portions as described in U.S. Pat. No. 7,549,982, filed Nov. 21, 2003, entitled “Tampon with Recessed Portions Having Multiple Widths,” issued to Carlin. The tampon may contain adjacent wide and narrow portions as described in U.S. Pat. No. 6,939,340, filed May 21, 2004, entitled “Tampon with Adjacent Wide and Narrow Raised Portions,” issued to Berges. The tampon may have one or more longitudinal grooves. The longitudinal grooves may be located in the insertion end, the withdrawal end, or both the withdrawal and insertion ends. The grooves may be offset as described in U.S. Pat. No. 8,029,485, filed Feb. 2, 2005, entitled “Tampon with Offset Grooves,” issued to Jensen.

The tampon may be a non-layered, uniform structure, or it may be a laminar structure comprised of integral or discrete layers, or the tampon may have a folded structure, or it may be rolled, or any other of the structures which are known in the art. Generally, the tampon herein has to have a certain minimal rigidity, to facilitate the expulsion through the film cap. An additional patch may be located between the absorbent compressed member and the overwrap as disclosed in U.S. Pat. No. 8,048,053, filed Apr. 14, 2008, entitled “Tampon Having an Auxiliary Patch,” issued to Minoguchi, et al.

The tampon may be constructed from a wide variety of liquid-absorbing materials commonly used in absorbent articles such as rayon, cotton, or comminuted wood pulp which is generally referred to as airfelt. Examples of other suitable absorbent materials include creped cellulose wadding; meltblown polymers including coform; chemically stiffened, modified or cross-linked cellulosic fibers; synthetic fibers such as crimped polyester fibers; peat moss; foam; tissue including tissue wraps and tissue laminates; or any equivalent material or combinations of materials, or mixtures of these. Preferred absorbent materials comprise cotton, rayon (including tri-lobal and conventional rayon fibers, and needle punched rayon), folded tissues, woven materials, nonwoven webs, synthetic and/or natural fibers. The tampon and any component thereof may comprise a single material or a combination of materials. Acceptable types of rayon include GALAXY Rayon (a tri-lobed rayon structure) available as 6140 Rayon from Acordis Fibers Ltd., of Hollywall, England and SARILLE L rayon (a round fiber rayon), also available from Acordis Fibers Ltd. Suitable cotton material includes, long fiber cotton, short fiber cotton, cotton linters, T-fiber cotton, card strips, and comber cotton. Preferably, the cotton layers should be a scoured & bleached cotton absorbent with a glycerin finish, a lemolin finish, or other suitable finish. Additionally, superabsorbent materials, such as superabsorbent polymers or absorbent gelling materials may be incorporated into the tampon.

The absorbent material may be surrounded with an overwrap. The overwrap may have liquid permeable material, if desired. Such materials may comprise rayon, cotton, bicomponent fibers, or other suitable natural or synthetic fibers known in the art. Rayon, polyethylene, polypropylene and blends of these are particularly suited for use as cover material. he synthetic fibers may include, but are not limited to, fibers such as polyester, polyolefin, nylon, polypropylene, polyethylene, polyacrylic, cellulose acetate or bicomponent fibers. Natural fibers may include, but are not limited to, those commonly known to be non-synthetic and of natural origin such as cotton and/or rayon. In general, the natural fibers may provide ready absorption and fluid wicking strength. The synthetic fibers may balance the capillary strength of the blended material, enabling the tampon to more readily slip against moist tissue, resulting in easier removal and hence removal comfort. The overwrap may be fluid wicking and may extend beyond the withdrawal end of the abosrobent material to form a skirt portion as described in U.S. Pat. No. 6,840,927, filed Nov. 16, 2001, entitled “Tampon with Fluid Wicking Overwrap With Skirt Portion,” issued to Hasse, et al. Typically, the overwrap may extend from about 2 mm to about 30 mm beyond the withdrawal end of the absorbent material.

The ratio of synthetic fibers to natural fibers may fall in the range of from about 90:10 to about 30:70. Alternatively, the ratio of synthetic fibers to natural fibers may fall in the range of from about 70:30 to about 40:60. The synthetic fibers may have hydrophobic and/or hydrophilic surfaces. The synthetic fibers may be inherently hydrophilic, or may preferably be treated to provide such properties. The overwrap may comprise some level of hydrophobic fibers as well, as long as it does not significantly diminish the fluid wicking capacity of the overwrap of the tampon.

The blend of fibers forming the overwrap may be made by any number of techniques. The blends may be carded on webs. Commonly, carded webs that are hydroentangled, thermally bonded, and resin bonded all have application. In the latter case, the resin bonding agent may be used in place of the synthetic fibers as the method for tempering the aggressiveness of the natural fiber matrix. In this case, all natural fiber may be used with a significant amount of synthetic binder (10-30% by weight is common). Spunbond and meltblown processes, combining synthetic fibers extruded/spun onto/into a mat or carded web of natural fibers provide other acceptable techniques. The basis weight of the overwrap may fall into a range from about 10, 12 or 15 grams per square meter to about 30, 40, 50 or 60 grams per square meter. The materials for the tampon may be formed into a fabric, web, or batt that is suitable for use in the pledget by any suitable process such as airlaying, carding, wetlaying, or other known techniques.

Fluid pervious overwrap may be made by any number of known techniques, but is preferably an apertured nonwoven material. The nonwoven material may be made by carding, meltblowing, spunbonding, spunlacing, air laying, and the like. The apertures may be zoned as described in U.S. Pat. No. 7,994,387, filed on Oct. 17, 2007, entitled “Tampon having Zoned Apertured Overwrap,” issued to Minoguchi, et al. In one embodiment, the apertures are formed by forming a plurality of spaced, melt stabilized regions, and then ring-rolling the web to stretch the web and form apertures in the melt stabilized regions, as described in U.S. Pat. Nos. 5,628,097 and 5,916,661, both of which are hereby incorporated by reference herein.

It is desirable that the tampons are made in the absorbency ranges, which are currently required, by the United States Food and Drug Administration and corresponding agencies of many other governments, which regulate tampon absorbency. A “Super Plus” absorbency tampon should have a total absorbency as measured by the industry standard Syngyna test of 12-15 grams. A “Super” absorbency tampon should have a total absorbency as measured by the Syngyna test of 9-12 grams. A “Regular” absorbency tampon should have a Syngyna absorbency of 6-9 grams. A “Junior” absorbency tampon should have a Syngyna absorbency of less than 6 grams. Providing a tampon which properly falls within these absorbency ranges requires that the total amount and type of absorbent material be controlled.

The tampon typically contains a withdrawal cord or string, which is generally attached to at least the withdrawal bottom side of the tampon. This may be any type of withdrawal cord known in the art, for example a generally braided (or twisted) withdrawal cord. A conventional type of withdrawal cord (in terms of thickness, material composition, etc.) may be periodically braided with a thicker slub of absorbent fibrous material, which acts as an absorbing member, to form a structure to be connected to the remaining of the tampon. In such an embodiment, the portion of the cord, which will act as the withdrawal cord, may be treated to make it non-absorbent or even hydrophobic. It may also be a withdrawal cord as described in commonly assigned and co-pending U.S. application Ser. No. 9/309,467, filed on May 10, 1999 in the name of Taylor, et al. The tampon may contain any additional functional ingredients, such as antimicrobial agents, lubricants, antioxidants etc, as known in the art.

The tampon and applicator may be placed inside a wrapper or wrapper material. By ‘wrapper material’ it is meant herein any material suitable to be used for hygienically wrapping tampons. Said wrapper material has two surfaces; the ‘inner surface’ is directed towards the wrapped tampon, whereas the ‘outer surface’ is aligned opposite to said inner surface. Typically, suitable wrapper materials for use herein are flexible polymeric films, having a thickness of less than 1 mm Examples for wrapper materials suitable for use are polymeric films made of polyethylene, polypropylene, polyester, cellophane, polyamide, poly(vinyl chloride), ethylene-vinyl acetate copolymer and the like. Alternatively, heat-shrinkable films, stretch films, pre-stretched elastic material, or combinations thereof may be used to create the wrapper. While not limited to a given composition, preferred compositions of heat-shrinkable and stretch films comprise primarily polyolefins such as polyethylene and polypropylene, or polyvinyl chloride. Polystyrene and polyethylene-terephtalate (PET), although being not heat sealable, are also suitable for use. Wrappers consisting of those materials can be closed by gluing with an adhesive. Other generally occlusive materials include metallic foils, such as aluminium foil. While occlusive wrapper materials are often preferred, in other situations non-occlusive or porous materials can be used, such as nonwovens, wovens, scrims, meshes and papers. Such non-occlusive materials can be made occlusive by combinations such as by lamination with or by coating with occlusive material. In the case of cellulosic papers, examples include lamination with a polymeric film such as a polyolefinic composition or coating or impregnation of the paper with wax. The aforementioned materials can be coated with various chemical compounds to improve their barrier properties or the ability for sealing. The wrapper may have a line of weakness or an improved opening means as described in U.S. Pat. No. 6,955, 665, filed May 23, 2002, entitled “Tampon Wrapper with Improved Opening Means,” issued to Domeier, et al. or U.S. Pat. No. 8,302,844, filed Nov. 20, 2006, entitled “Wrapper Having a Predetermined Line of Weakness,” issued to Mc Connell, et al.

Procedure to measure Onset Crystallization Temperature

Use a Differential Scanning Calorimeter (DSC) according to ASTM D3418. Follow ASTM D3418 to calibrate the DSC instrument and to prepare the sample. Using the following modifications, determine the onset temperature of the crystallization peak (OTX) in ° C. for the sample using the following temperature program.

1. Equilibrate the sample at 200° C. to erase previous thermal history.

2. Hold the sample at 200° C. for 5 minutes.

3. Ramp the sample to 120° C. at a rate of 50° C. per minute.

4. Hold the sample at 120° C. for 1 minute to stabilize the temperature.

5. Ramp the sample to 90° C. at a rate of 2° C. per minute.

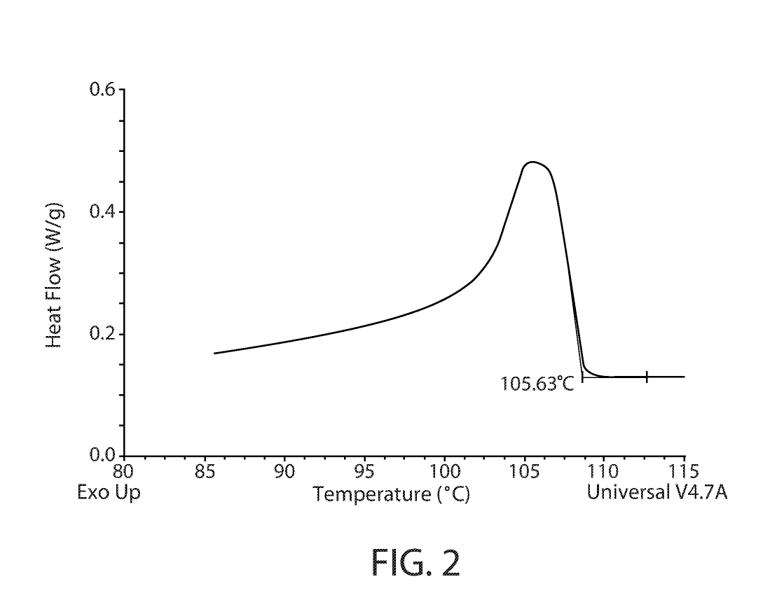

For each sample, use the DSC software to determine the intersection of two asymptotes, as shown in FIG. 2. Record the onset temperature crystallization peak (OTX) to the nearest 0.01° C. Calculate OTX for the 3 replicates and report the average OTX to the nearest 0.01° C.

Tampon Applicator Opacity, Color and Color Masking

All opacity, color and color masking measurements are made using a 0°/45° spectrophotometer suitable for making standard CIE L*a*b* color measurements (e.g. Hunter Labscan XE spectrophotometer, Hunter Associates Laboratory Inc., Reston VA or equivalent). The diameter of the instrument's measurement port should be smaller than the dimensions of the sample. Analyses are performed in a room controlled at about 23° C.±2° C. and 50%±2% relative humidity. Samples are conditioned at the same condition for 2 hours before testing.

Samples are prepared by first removing and discarding the pledget and plunger from the barrel. Using an Exacto knife, the finger-grip portion as well as the petal portion of the barrel is removed. The barrel is then cut open with a longitudinal slice from the petal end to the finger-grip region. The sample should be free from creases, wrinkles, tears, and other obvious defects. For all testing, the sample is positioned so that the outer surface of the applicator as it is converted will be the surface of the sample that faces the orifice of the instrument sample port. The sample is positioned so that it lies flat and the longitudinal centerline between the petal and finger-grip regions is centered over the spectrophotometer orifice.

Opacity is measured by contrast ratio. Calibrate the instrument per the vendor instructions using the standard black and white tiles provided by the vendor. Set the spectrophotometer to use the CIE XYZ color space, with a D65 standard illumination, a 10° observer and the UV filter set to nominal. Place the sample flat against the instrument's measurement port and then place the white standard tile onto the opposing surface of the sample such that it completely covers the measurement port. Take a reading for XYZ and record to 0.01 units. Without moving the sample, remove the white tile and replace it with the black standard tile. Take a second reading for XYZ and record to 0.01 units. Repeat this procedure for a total of three (3) replicate samples. Opacity is calculated by dividing the Y value measured using the black tile as backing, divided by the Y value measured using the white tile as backing, then multiplying the ratio by 100. Record the opacity value to the nearest 0.01%. Calculate opacity for the 3 replicates and report the average opacity to the nearest 0.01%.

The color scale values, utilized herein to define the darkness/lightness of the tampon applicator material according to the present invention, is the widely accepted CIE LAB scale. Measurements are made on the sample directly after measuring the opacity. Set the spectrophotometer to use the CIE LAB color space, with a D65 standard illumination, a 10° observer and the UV filter set to nominal. Color measurements are made on the sample with the white standard tile as backing. Place the sample flat against the instrument's measurement port and then place the white standard tile onto the opposing surface of the sample such that it completely covers the measurement port. Take a reading for L*, a*, b* and record to 0.01 units. Repeat this procedure for a total of three (3) replicate samples. Report the average L*, a*, b* values to the nearest 0.01 units.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm ”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of designing a feminine hygiene applicator, the method comprising the steps of:
obtaining a first applicator;
obtaining onset crystallization temperature for the first applicator;
obtaining a second applicator;
obtaining L*, a*, b* values for the second applicator;
configuring a third applicator comprising an onset crystallization temperature which is within 3 percent of the onset crystallization temperature of the first applicator;
configuring the third applicator to have an L* value within 20 percent of the L* value for the second applicator;
configuring the third applicator to have an a* value within 20 percent of the a* value for the second applicator; and
configuring the third applicator to have a b* value within 20 percent of the b* value for the second applicator.

2. The method of designing a feminine hygiene applicator of claim 1, wherein the opacity of each of the first applicator and the second applicator is greater than 41% and less than 75%.

3. The method of designing a feminine hygiene applicator of claim 1, wherein the plungers for each of the applicators of the first product and second product are unitary.

4. The method of designing a feminine hygiene applicator of claim 1, wherein the plungers for each of the applicators of the first product and second product comprise an outer sleeve and an inner sleeve, wherein the inner sleeve is slidingly engaged with the outer sleeve.

5. The method of designing a feminine hygiene applicator of claim 1, wherein the first feminine care product is selected from the group comprising tampons and pessaries.

6. The method of designing a feminine hygiene applicator of claim 1, wherein the onset crystallization temperature of each of the first applicator and the second applicator is less 109 degrees Celsius.

7. The method of designing a feminine hygiene applicator of claim 1, wherein the onset crystallization temperature of each of the first applicator and the second applicator is greater than 107 degrees Celsius.

8. The method of designing a feminine hygiene applicator of claim 1, wherein the first applicator barrel portion comprises the first color and wherein the first applicator plunger comprises a third color.

9. The method of designing a feminine hygiene applicator of claim 1, wherein the first color comprises an a* between −128 and 128 and a b* between −128 and 128, wherein the second color comprises an a* between −128 and 128 and a b* between −128 and 128, and wherein the first color a*and the second color a* differ by less than 20.

10. The method of designing a feminine hygiene applicator of claim 1, wherein the first product and the second product are offered from same manufacturer and wherein the first color is not equal to the second color.

11. The method of designing a feminine hygiene applicator of claim 9, wherein the first applicator is offered for sale during a first time period and the second applicator is offered for sale during a second time period, wherein the first time period and the second time period are not the same.

12. The method of designing a feminine hygiene applicator of claim 1, wherein the first applicator comprises an onset crystallization temperature of about 107+_0.4 degrees Celsius, an opacity of greater than 50%, and an a* of negative 100 to positive 100.

13. The method of designing a feminine hygiene applicator of claim 9, wherein the first color a* is between 0 and 128.

14. A method of designing a feminine hygiene applicator, the method comprising the steps of:
obtaining a first applicator;
obtaining onset crystallization temperature for the first applicator;
obtaining a second applicator;
obtaining L*, a*, b* values and an opacity value for the second applicator;
configuring a third applicator comprising an onset crystallization temperature which is within 3 percent of the onset crystallization temperature of the first applicator;
configuring the third applicator to have an L* value within 20 percent of the L* value for the second applicator or an a* value within 20 percent of the a* value for the second applicator and/or a b* value within 20 percent of the b* value for the second applicator; and
configuring the third applicator to have an opacity value which is less than that of the opacity value for the second applicator.

15. The method of designing a feminine hygiene applicator of claim 14, wherein the each of the first applicator and the second applicator have an opacity and wherein the opacity of each of the first applicator and the second applicator is greater than 41 and less than 75.

16. The method of designing a feminine hygiene applicator of claim 14, wherein the plungers for each of the applicators of the first product and second product are unitary.

17. The method of designing a feminine hygiene applicator of claim 14, wherein the plungers for each of the applicators of the first product and second product comprise an outer sleeve and an inner sleeve, wherein the inner sleeve is slidingly engaged with the outer sleeve.

18. The method of designing a feminine hygiene applicator of claim 14, wherein the first feminine care product is selected from the group comprising tampons and pessaries.

19. The method of designing a feminine hygiene applicator of claim 14, wherein the applicator for the first product and the applicator for the second product are configured the same.

20. The method of designing a feminine hygiene applicator of claim 14, wherein the opacity of the applicator for the first product is between 60 and 80 and the opacity of the applicator for the second product is between 40 and 55.
